(19) United States
(12) Patent Application Publication
(10) Pub. No.: US 2024/0238028 A1
(43) Pub. Date: Jul. 18, 2024

(54) METHOD AND SYSTEM OF REMOVING VISCERAL FAT

(71) Applicant: B2M MEDICAL, INC., Irvine, CA (US)

(72) Inventors: Meital MAZOR, San Diego, CA (US); Alexei V. Babkin, Dana Point, CA (US); Rafi MAZOR, San Diego, CA (US)

(73) Assignee: B2M MEDICAL, INC., Irvine, CA (US)

(21) Appl. No.: 18/562,292

(22) PCT Filed: May 23, 2022
(86) PCT No.: PCT/US22/72511

§ 371 Date: Nov. 18, 2023

Related U.S. Application Data
(60) Provisional application No. 63/192,545, filed on May 24, 2021.

(51) Int. Cl. A61B 18/02; A61B 18/00

(52) U.S. Cl. CPC A61B 18/02; A61B 2018/00035; A61B 2018/00041; A61B 2018/00464; A61B 2018/0293; A61B 2218/007

(57) ABSTRACT

A method of reducing visceral fat in the body of a patient comprises thermally treating the visceral fat sufficient to form a liquid-fat mixture or emulsion. The liquid-fat emulsion is then aspirated. Thermally treating can be performed by cooling according to a temperature profile causing the cell membranes to be disrupted. The visceral fat cells are warmed with a warming liquid immediately following cooling. The warming liquid, along with the contents of the fat cells that mix with the warming liquid, are withdrawn by aspiration.

### CROSS REFERENCE TO RELATED APPLICATIONS

This application claims priority to application No. 63/192,545, filed May 24, 2021, entitled “METHOD OF REMOVING VISCERAL FAT.”

### FIELD OF THE INVENTIONS

The inventions described below relate to the field of visceral fat reduction.

### BACKGROUND OF THE INVENTIONS

Visceral fat is found inside the abdominal cavity and wraps around internal organs, as opposed to subcutaneous fat which is stored just below the skin. Visceral fat may be found in the abdomen, under the abdominal muscles. Visceral fat is associated with high blood pressure, increased risk of heart disease, insulin resistance and diabetes, stroke, some cancers, and continued presence in the body may contribute to these conditions.

Various approaches have been attempted to treat visceral fat. Diet and exercise can help eliminate visceral fat. However, diet and exercise are not well-tolerated by the typical overweight patient.

Visceral fat can also be destroyed by cooling to temperatures in the range of +10 C to −40 C. If cooling is limited to this range, surrounding tissue will not be killed. Cryogenically deadened visceral fat will typically be resorbed by the body over the course of a few weeks.

The mechanism of cell death of cooled visceral fat has not been definitively established. It may be due to lysis (disruption of the cell walls), necrosis (death of the cell without disruption of the cell walls) or apoptosis (programmed cell death after injury due to cooling). In prior art methods, regardless of the mechanism of cell death, dead or dying visceral fat cells are left in the body to be resorbed over a period of weeks. This is undesirable.

Liposuction is another treatment to remove fat. Liposuction mechanically disrupts the fat cells and surrounding extracellular matrix, blood vessels, and subsequent aspiration. However, liposuction is relatively violent and limited to the subcutaneous fat.

Accordingly there is still a need for an improved method and system that overcomes the above mentioned shortcomings.

### SUMMARY OF THE INVENTION

In embodiments of the present invention, a method of reducing visceral fat from a body of a patient comprises thermally treating the visceral fat in the body of the patient sufficient to form a liquid-fat mixture or emulsion. The liquid-fat emulsion is then aspirated.

In embodiments, the treating comprises cooling the visceral fat in situ to a low temperature sufficient to cause the visceral fat cells to freeze, and for the cell membranes to be disrupted, burst or rupture. The low temperature can range from +10 to −40 degrees C. The cooling can be performed for a duration between 10 and 60 seconds, and more preferably, for less than 30 seconds.

In embodiments, cooling is halted for a period of time to allow the fat cells to warm, releasing the contents of the fat cells, thereby forming a liquid-fat mixture or emulsion.

In embodiments, the treating further comprises delivering a warming liquid into the body of the patient at a warm temperature sufficient to thaw the fat cells, thereby releasing contents including lipids into the warming liquid and forming the liquid-fat emulsion. In preferred embodiments, the warming liquid is saline.

In embodiments, the cooling step is performed with a cooling device comprising a handle, a shaft, and a distal treatment section adapted to conduct heat from the visceral fat to cool the visceral fat to the low temperature.

In embodiments, the delivering a warming liquid is performed with a liquid delivery device. The delivery of the warming liquid can be performed for a duration ranging from 1 to 5 minutes within five (5) minutes from the cooling step, or optionally, within two (2) minutes from the cooling step. In embodiments, the warm temperature ranges from 25 to 37 degrees C.

In embodiments, the aspiration step is performed with an aspiration device, and optionally, the aspiration device is integrated with the liquid delivery device.

In embodiments, the method further comprises measuring temperature, and optionally, measuring temperature with a temperature sensor along the distal treatment section of the cooling device. The cooling device can be adjusted or controlled based on the measured temperature.

In embodiments, the treatment is performed on visceral fat, and preferably visceral fat in the mesentery.

In embodiments, the method further comprises visualizing the treatment of the visceral fat, and optionally, wherein the visualizing is performed with a laparoscope.

In embodiments, a surgical system for reducing visceral fat from a body of a patient comprises a cooling device comprising a distal treatment section adapted to conduct heat from the visceral fat to cool the visceral fat; a fluid delivery device for immersing the cooled visceral fat in a warm liquid; and an aspiration device adapted to withdraw the liquid-fat emulsion from the body of the patient.

In embodiments, the system further comprises a controller programmed and operable to (a) control the cooling device according to a cooling temperature profile sufficient to cause the visceral fat cells to freeze and to disrupt the fat cell membrane, and (b) optionally, to control the fluid delivery device to control the warm liquid according to warming temperature profile sufficient to thaw the fat cells, thereby releasing lipids from the fat cells into the warming liquid and forming a liquid-fat mixture or emulsion.

The description, objects and advantages of embodiments of the present invention will become apparent from the detailed description to follow, together with the accompanying drawings.

### BRIEF DESCRIPTION OF THE DRAWINGS

FIG. 1 illustrates the anatomy of a patient, showing one location of visceral fat within the body, and the cooling of the visceral fat with a cooling probe in accordance with an embodiment of the invention.

FIG. 2 illustrates the liquefaction of the visceral fat in the deposit shown in FIG. 1, and the aspiration of the liquid fraction from the body in accordance with an embodiment of the invention.

FIG. 3 is a flow chart illustrating a method for removing visceral fat in accordance with an embodiment of the invention.

FIG. 4 is a block diagram illustrating a visceral fat removal system accordance with an embodiment of the invention.

### DETAILED DESCRIPTION OF THE INVENTIONS

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

All existing subject matter mentioned herein (e.g., publications, patents, patent applications and hardware) is incorporated by reference herein in its entirety except insofar as the subject matter may conflict with that of the present invention (in which case what is present herein shall prevail). The following are incorporated by referenced in their entirety for all purposes: Methods and systems for cooling visceral fat while leaving surrounding tissue unharmed are disclosed in our prior International Patent Publication WO 2020/061202 (published Mar. 26, 2020) and International Patent Publication WO 2021102301 (published May 27, 2020).

Without intending to being bound to theory, in our own studies, we have observed significant liquefaction of visceral fat cells when the fat cells are cooled to the range of −20° C. to −40° C., and thawed. The lipids from the cells appear loose within the tissue, and at a volume and viscosity susceptible to removal by aspiration. This makes it feasible to remove some of the mass of the fat cells, such as this free liquid fraction, without mechanical disruption of the fat cells, by aspiration.

In embodiments, a method of removing visceral fat from the body of a patient comprises the steps of: cooling visceral fat within the body of a patient to a temperature sufficient to cause liquefaction of the visceral fat cells; allowing the cooled visceral fat to warm to a low viscosity susceptible to aspiration, which may be below body temperature or at body temperature (or actively warm it with a warming function of the probe); aspirating a liquid fraction caused by cooling from the body, from the site of liquefaction; avoiding mechanical disruption of the visceral fat and extracellular matrix while performing the cooling and aspiration steps; leaving any remaining solid portion of the visceral fat in the body to be resorbed by the body; and leaving the extracellular matrix and/or blood vessel within the visceral fat in the body.

The step of cooling visceral fat within the body of a patient to a temperature sufficient to cause liquefaction of the visceral fat cells may be accomplished with various cooling methods, examples of which are described herein. Modes may include closed loop or open loop cooling probes or application of cooling packs or even flushing fat with a cold slurry, gas or liquid. A wide range of types of cooling devices (including any cryoprobe, catheter, instrument whether rigid or flexible) capable of cooling the visceral fat to temperatures in the desired range may be used, whether cooled with liquid cryogens or gaseous cryogens, or whether cooled by cold fluid (a gas, liquid or a mixture of gas and liquid, for example), or cooled by expanding gas (argon or nitrogen gas, for example). The cooling probe may be a long, small diameter probe such as a cryoprobe used for freezing tumors. The cooling probe may include expandable cooling structures such as a pad or balloon which may be compacted into an insertion cannula and expanded after insertion to expose a larger portion of visceral pad to cooling. The cooling probe may be inserted, needle-like, without any surrounding cannula or introducer, or a cannula or introducer may be first placed into the visceral fat to provide access, and the cooling probe may then be inserted through the cannula or introducer. This may be preferred if the aspiration step is accomplished with a separate aspiration catheter.

Cooling with a cooling probe is accomplished while controlling the cooling probe to cool visceral fat surrounding the distal end of the cooling probe to temperatures below about −20° C. Preferably, cooling is accomplished while controlling the cooling probe to cool visceral fat surrounding the distal end of the cooling probe to temperatures in the range of −20° C. to −40° C. and controlling the cooling probe to avoid cooling the visceral fat surrounding the distal end of the cooling probe to temperatures below about −40° C., to avoid cryogenic damage to organs, blood vessels and nerves within, or near, the visceral fat.

For visceral fat near the intestines, cooling with a cooling probe may be accomplished while controlling the cooling probe to cool visceral fat surrounding the distal end of the cooling probe to temperatures in the range of about −20° C. to −30° C., and controlling the cooling probe to avoid cooling the visceral fat surrounding the distal end of the cooling probe to temperatures below about −30° C., to avoid cryogenic damage to intestines, which may be more susceptible to damage at colder temperatures.

For large deposits of visceral fat, with some distance from organs, cooling with a cooling probe may be accomplished while controlling the cooling probe to cool visceral fat surrounding the distal end of the cooling probe to temperatures in the range of about −20° C. to −60° C., preferably −40 to −20° C. and controlling the cooling probe to avoid cooling the visceral fat surrounding the distal end of the cooling probe to temperatures below about −60° C., again to avoid cryogenic damage to organs while speeding the cooling process.

In open procedures, flushing the visceral fat with cold fluid such as nitrogen gas may be used to cool the visceral fat. Cooling for a short period of about 30 to 40 seconds has proven sufficient to cool to the temperature ranges mentioned above, but cooling for longer periods may result in a larger zone of liquefaction without harming nearby organs, blood vessels or nerves.

After cooling, the visceral fat may be warmed to a temperature at which liquefaction is evident and the liquid fraction is released from the fat cells. Warming may be passive, using the patient's body temperature to warm the visceral fat, or the warming may be active, using any means for actively warming the visceral fat such as warming fluid flowing through the probe (warm water, or using expanding helium in a Joule-Thompson probe), heating wires within the probe, or RF or microwave energy delivered through the probe.

Upon warming of the visceral fat tissue after cooling, the liquid fraction becomes visible and has a viscosity low enough to remove by aspiration. When this point is reached, the liquid fraction of the visceral fat released by the cooling step is aspirated from the body. Aspiration may be accomplished through the cooling probe, if fitted with an aspiration lumen so that it may be used as an aspiration tube (a catheter, cannula or needle) or with a separate aspiration tube (a catheter, cannula or needle).

Because the visceral fat surrounds other organs and blood vessels and nerves, the method is preferably accomplished without using a means to mechanically disrupt the visceral fat.

The method is preferably accomplished without using the cooling probe, or the separate warming probe, or any other component inserted into the body, to mechanically disrupt the visceral fat (beyond the slight disruption inherent in pushing the cooling probe against the visceral fat). Thus, the method is preferably accomplished without reaming, macerating, dissecting, resecting the visceral fat, or manipulating the cooling probe in direction other than longitudinal translation along the axis of the probe for insertion and removal. The method can be characterized as a gentle or atraumatic thermal treatment.

As the liquid fraction is traveling along the aspiration catheter and tubing, it may cool to ambient, from body temperature, and become more viscous and semi-solid (like butter). Thus, the aspiration catheter and tubing may be provided with a means for warming the catheter and/or associated tubing, such as heating wires or warm water circulation in a jacket surrounding the aspiration catheter. The distal end of the aspiration catheter may be left without means for warming, as it is disposed within the body when in use. Again, the aspiration step is preferably accomplished without reaming, macerating, dissecting, resecting the visceral fat, or manipulating the aspiration catheter in direction other that longitudinal translation along the axis of the probe for insertion and removal.

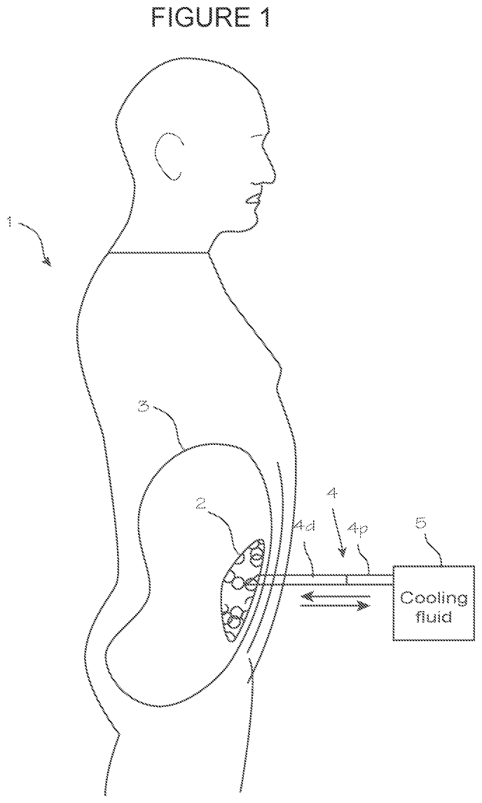

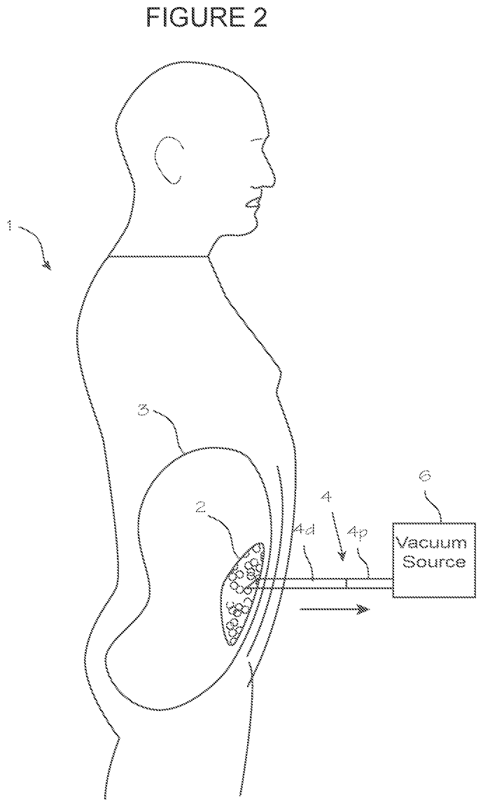

FIGS. 1 and 2 illustrate a method in accordance with embodiments of the invention. FIG. 1 shows a patient 1 and a deposit of visceral fat 2 within the patient's abdomen/peritoneum 3. An insertion portion 4d of cooling probe 4 has been inserted into the abdomen/peritoneum and into visceral fat deposit 2. The proximal end 4p of the cooling probe is connected in fluid communication with a cooling fluid source 5. The type of cooling probe and cooling source may vary. Examples are described in International Patent Publication WO 2020/061202 (published Mar. 26, 2020) and International Patent Publication WO 2021102301 (published May 27, 2020).

FIG. 2 illustrates use of the cooling probe 4 as an aspiration catheter, with the insertion portion remaining in the visceral fat deposit 2 after cooling and warming has been accomplished, as described above. The probe 4 is shown connected in fluid communication with a vacuum source 6, which is operable to apply vacuum to the probe and aspirate the liquid fraction of the cooled and warmed visceral fat.

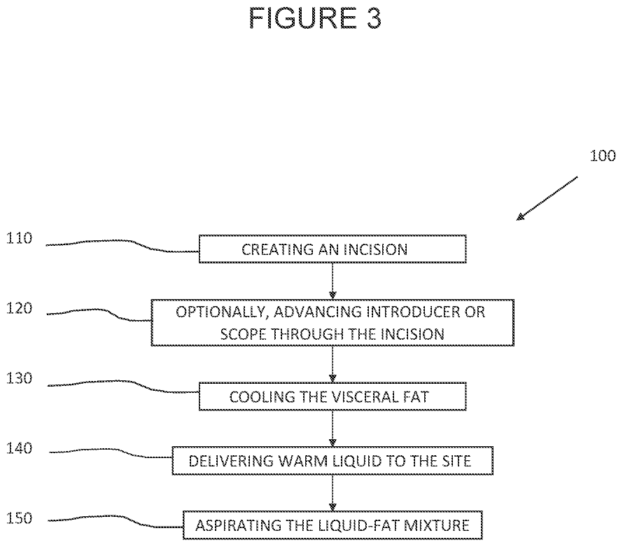

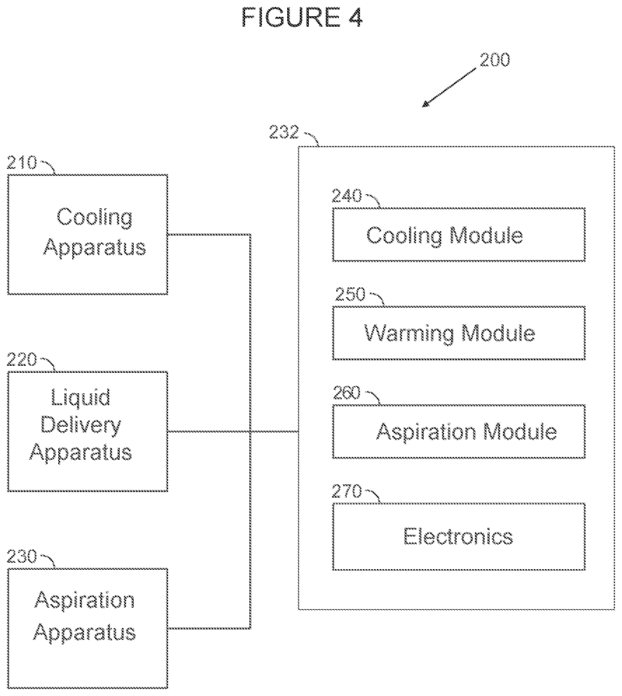

FIG. 3 illustrates another method 100 for reducing visceral fat in the body of a patient in accordance with an embodiment of the invention. As the steps of the method are described herein, reference is also made to the illustrations shown in FIGS. 1-2, and the system block diagram shown in FIG. 4 to show non-limiting exemplary components for carrying out each of the steps.

Step 110 states creating an incision. In embodiments, an incision is made in the skin of the patient to provide access to the abdomen cavity and the visceral fat. In embodiments, the incision is a small keyhole incision suitable for a laparoscope and minimally invasive surgery. In other embodiments, a laparotomy is performed suitable for an open surgery.

Step 120 states advancing an introducer or scope, namely, a laparoscope into the body of the patient. If the physician determines to reduce the visceral fat using a minimally invasive approach, an introducer or perhaps laparoscope is advanced through the incision and the additional treatment steps are performed in combination through the laparoscope or introducer.

However, this step 120 is optional and in embodiments, the incision is made sufficiently large to provide access to the visceral fat, and step 120 is omitted.

Step 130 states cooling the visceral fat. This step may be performed using a cooling probe 4 or a cooling apparatus 210 as described herein and as shown in FIGS. 1 and 4, respectively.

The cooling probe or apparatus may be couplable to a controller or console 232 via an umbilical cord. With reference to FIG. 4, the controller or console 200 may include a cooling module 240 and computer electronics 270 to provide cooling power to the cooling apparatus 210. The cooling module 240 may comprise various components (e.g., cooling supply or source, refrigeration unit, valves, etc.). The computer electronics 270 may comprise those types of electronics typically found in a computer including, e.g., a power supply, PCB, memory, communication module, ethernet connection, input ports, graphics and video card modules, and a processer operable to carry out the treatment steps described herein. In preferred embodiments, the cooling module is operable to provide a cooling temperature profile sufficient to cool the visceral fat between −20 and −60 degrees C. for a duration of 10-60 seconds, and more preferably 5-30 seconds. This may include a set of computer readable instructions or software operable with the processor to turn on and off the cooling apparatus, control its temperature (optionally, control its temperature based on real time temperature measurements from the cooling probe), and alert the user to ready status, cooling completed, temperature out of range, service required, etc. Non-limiting examples of cooling devices and their components are described in International Patent Publication WO 2020/061202 (Mar. 26, 2020) and International Patent Publication WO 2021102301 (May 27, 2020).

In embodiments, and without intending to being bound to theory, the cooling causes the contents within the fat cells to freeze and expand, causing the cell membranes to be disrupted, burst or rupture. Upon thawing, discussed herein, these frozen contents including lipids and liquids are freed.

Step 140 states delivering warm liquid to the site. In a sense, the site is flooded with warm liquid. This step may be performed using the cooling probe 4 when fitted with a fluid delivery channel, or with a separate liquid delivery apparatus 220 as shown in FIGS. 1 and 4, respectively. The separate liquid delivery apparatus may be couplable to the controller 232 via an umbilical cord. The delivery apparatus may include a liquid source such as an elevated IV bag and valve, reservoir, syringe, or fluid supply and pump. In a preferred embodiment, and with reference to FIG. 4, console 200 includes a warming module including various components such as a fluid supply, pump, heater, temperature sensor, software operable to carry out the fluid delivery steps described herein. In preferred embodiments, the warming module 250 is operable to provide a warming temperature profile sufficient to warm the visceral fat between 20 and 40 degrees C. for a duration of 3-10 minutes, and more preferably 3-5 minutes. Additionally, the warming module 250 or controller 232 is operable commence the warming cycle immediately subsequent to the termination of the cooling cycle, or within 1 minute from the cooling cycle.

A minimum volume delivered can be set by the warming 250 to ensure the viscosity mixture is in a range allowing for aspiration. The volume delivered can be monitored based on flowrate, duration, and the controller can halt delivery when a set volume is reached. In embodiments the set volume or maximum volume delivered is limited by the controller to range from 500 to 5000 ml per segment of visceral fat treated.

This may include a set of computer readable instructions or software operable with the processor to turn on and off the fluid delivery apparatus, control its liquid temperature based on real time temperature measurements at one or more locations along the liquid path, control the flowrate of the fluid delivered to the target area, and alert the user to ready status, temperature out of range, time elapsed, service required, etc.

As described herein, the warming liquid serves to warm and thaw frozen cell contents. Upon thawing, these frozen contents including lipids and liquids are freed from the cell. The delivered warm liquid takes up and mixes with the freed cell components, forming a liquid-fat mixture or emulsion. The delivered warming liquid thus serves as a cryo-lipolysis vehicle to accept and transport the lysed matter.

Step 150 states aspirating the liquid-fat mixture. This step may be performed using the cooling probe 4 when fitted with an aspiration channel, or with a separate aspiration apparatus 230 as shown in FIGS. 1 and 4, respectively. The separate aspiration apparatus 230 may be an aspiration instrument or catheter couplable to the controller 232 via an umbilical cord. In embodiments, and in contrast to liposuction type devices, the aspiration apparatus 230 preferably comprises a soft or atraumatic tip incapable of cutting or tearing or morcellating tissue and optionally a flexible shaft having a modulus of elasticity sufficiently low such that the aspiration apparatus is incapable to injuring or tearing tissue. In embodiments, the aspiration apparatus is a flexible suction line with an atraumatic tip to facilitate aspiration without injuring the tissue.

In a preferred embodiment, and with reference to FIG. 4, console 200 includes an aspiration module 260 including various components such as a motor, collection reservoir, flow meter, power supply, temperature sensor, pressure sensor, optional heating elements to prevent the fat from solidifying along the aspiration flowpath, and software operable to carry out the aspiration steps described herein. In preferred embodiments, the aspiration module 260 is operable to provide a vacuum pressure sufficient to draw the liquid-fat emulsion or mixture and excess liquid. The module 260 may include a set of computer readable instructions or software operable with the processor to turn on and off the aspiration apparatus, control the suction applied and flowrate of the mixture being withdrawn, control evacuation lumen temperature, and alert the user to ready status, pressure out of range, time elapsed, estimated volume collected, service required, etc.

Not shown, the method may comprise displaying on a monitor a wide range of information including, for example, treatment parameters (e.g., cooling time, cooling temperature, thawing time, thawing temperature, aspiration time, number of applications, estimated mixture volume aspirated, estimated fat volume aspirated), status (e.g., offline, ready, cooling, thawing, aspirating, etc.), patient identification information, and a window for a laparoscope or camera view to visualize the treatment in real time.

### EXAMPLE

The following is an example procedure performed on an animal in accordance with an embodiment of the present invention.

Animal type: Ossabaw obese pig.

Fat type: Mesenteric (visceral).

Procedure: A laparotomy was performed to expose the small intestine's mesenteric fat. We used a cooling probe having a round head (2-3 inch in diameter) to spray gaseous nitrogen onto the target area. The temperature of the nitrogen gas was about −40° C. We applied a total of 40 applications (30 second each) to both sides of the mesenteric fat.

A warm saline solution (37° C.) was delivered to the area to thaw the fat following the cooling applications. Approximately 8 freezing cycles were applied to a segment of mesenteric fat before thawing with a warm (100-300 ml) saline solution. We treated the mesenteric fat and thawed with the saline as described above throughout the small intestine. When the freeze/thaw treatment was done, we aspirated the visible saline mixture from the pig which now included both the saline solution and “soluble fat” (or “liquid fat”).

Results: We collected the saline mixture and measured the amount of liquid fat that was removed at the time of the procedure (˜600 ml). We note this is an immediate and acute reduction to the mesenteric fat. The total amount of mesenteric fat in obese Ossabaw pigs is estimated at 3-6 L. Thus, a 600 ml reduction is an approximately ˜10-20% reduction of the overall fat in this anatomical storage region.

We would also expect further fat reduction over several weeks following cryo-lipolysis. Embodiments of the invention include a two phase approach comprising acute/immediate fat removal followed by gradual fat absorption following the initial immediate treatment. In embodiments, a first portion of the visceral fat is treated with an acute/immediate method (e.g., the method 100 described in connection with FIG. 3), and a second portion of the visceral fat (perhaps a persistent deep portion adjacent an obstacle or vessel) is identified for gradual absorption in which the fat is cooled to a lesser degree (e.g., between −10 to −25 deg. C.) than in the immediate treatment. Consequently, it is anticipated some of the fat cells of the persistent fat treated with the modified cooling therapy do not rupture but will still necrosis and be absorbed by the body over time. Additionally, any mixture not aspirated including lipids in the mixture shall be absorbed over time by the body. This process of absorption may range from 1-3 weeks.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. For example, the cooling apparatus, liquid delivery apparatus, and aspiration apparatus may be combined together as one instrument or may be separate discrete instruments. Other embodiments and configurations may be devised without departing from the spirit of the inventions and the scope of the appended claims.

1. A surgical system for reducing visceral fat from a body of a patient comprising: a cooling device comprising a distal treatment section adapted to conduct heat from the visceral fat to cool the visceral fat;
a fluid delivery device for rinsing the cooled visceral fat in a warm liquid;
a controller programmed and operable to
(a) control the cooling device according to a cooling temperature profile sufficient to cause the visceral fat cells to freeze and to disrupt the fat cell membrane, and
(b) optionally, to control the fluid delivery device to control the warm liquid according to warming temperature profile sufficient to thaw the fat cells, thereby releasing lipids from the fat cells into the warming liquid and forming a liquid-fat mixture or emulsion; and
an aspiration device adapted to withdraw the liquid-fat emulsion from the body of the patient.

2.-10. (canceled)

11. A method of reducing visceral fat from a body of a patient comprising thermally treating the visceral fat in the body of the patient sufficient to form a liquid-fat mixture or emulsion; and aspirating the emulsion from the patient.

12. (canceled)

13. The method of claim 11, wherein the treating comprises cooling the visceral fat within the body of the patient to a low temperature sufficient to cause the visceral fat cells to freeze.

14. The method of claim 13, wherein the treating further comprises delivering a warming liquid into the body of the patient at a warm temperature sufficient to thaw the fat cells, thereby releasing lipids into the warming liquid and forming the liquid-fat emulsion.

15. (canceled)

16. The method of claim 11, further comprising making an incision in the skin of the patient to access the visceral fat.

17. (canceled)

18. The method of claim 13, wherein the cooling step is performed with a cooling device comprising a handle, a shaft, and a distal treatment section adapted to conduct heat from the visceral fat to cool the visceral fat to the low temperature.

19. The method of claim 18, wherein the low temperature ranges from 0 to −40 degrees C.

20. The method of claim 14, wherein the delivering a warming liquid is performed with a liquid delivery device.

21. The method of claim 20, wherein the delivering a warming liquid is performed for a duration ranging from 1 to 5 minutes within five (5) minutes from the cooling step, or optionally, within two (2) minutes from the cooling step.

22. (canceled)

23. The method of claim 12, wherein the aspiration step is performed with an aspiration device, and optionally, the aspiration device comprises an aspiration lumen and a liquid delivery lumen for performing delivery of a warming liquid and aspiration of the liquid-fat emulsion.

24. The method of claim 18, further comprising measuring temperature with a cooling device, and wherein the cooling device is adjusted or controlled based on the measured temperature.

25. (canceled)

26. The method of claim 19, wherein the cooling is performed for a duration between 10 and 60 seconds.

27. The method of claim 11, wherein the visceral fat is in the mesentery.

28. The method of claim 11, further comprising visualizing the treatment of the visceral fat.

29. The method of claim 11, further comprising heating the liquid-fat mixture along a passageway out of the body to maintain the liquid-fat mixture in a low viscosity state.

30. The method of claim 18, further comprising advancing an introducer into the body of the patient to provide access to the visceral fat, and wherein the cooling device is advanced through the introducer.

31. The method of claim 11, comprising
avoiding mechanical disruption of the visceral fat and extracellular matrix while performing the cooling and aspiration steps.

32.-40. (canceled)

41. A surgical method for reducing fat cells in the body of a patient comprising accessing the fat cells through an incision the body of the patient and gently forming, in situ, a liquid-fat mixture from the fat cells.

42. The method of claim 41, comprising cooling the fat cells with a cooling apparatus such that cell membrane is disrupted, burst or ruptures.

43. The method of claim 42, further comprising delivering a liquid to the fat cells, wherein components of the cells are taken up by the liquid, thereby forming the liquid-fat mixture.

44. The method of claim 43, wherein the delivering step comprises warming or thawing the cooled cells by delivering a warm liquid.

45. The method of claim 44, further comprising aspirating the liquid-fat mixture.

46. The method of claim 45, wherein the fat cells are visceral fat cells.

47. The method of claim 46, wherein the fat cells are mesentery fat cells.
